(19) United States
(12) Patent Application Publication
(10) Pub. No.: US 2026/0198540 A1
(43) Pub. Date: Jul. 16, 2026

(54) Natural Salt-Reducing Peptide Composition and Its Use

(71) Applicant: Suzhou Aquafarmtory Biotechnology Co., Ltd., Suzhou (CN)

(72) Inventors: Weiwei Zhang, Suzhou (CN); Yang Liu, Suzhou (CN)

(21) Appl. No.: 19/442,840

(22) Filed: Jan. 7, 2026

(30) Foreign Application Priority Data
Jan. 7, 2025 (CN) 202510023551.8

(51) Int. Cl. A23L 27/21; A23L 27/00; C07K 7/06; C07K 14/00

(52) U.S. Cl. CPC A23L 27/21; A23L 27/88; C07K 7/06; C07K 14/001

(57) ABSTRACT

A natural salt-reducing peptide composition comprises at least two salt-reducing peptides, wherein both peptides are capable of binding to the TMC4 receptor and/or the ENaC receptor. The natural salt-reducing peptide composition provides a sensory experience similar to that of salt in terms of saltiness perception. It reduces salt concentration without diminishing perceived saltiness. As a future food additive, it enhances the health attributes of foods while meeting consumer expectations for flavor.

### CROSS-REFERENCE TO RELATED APPLICATIONS

The present application claims the priority to Chinese Serial No. 2025100235518, filed with Chinese Patent Office on Jan. 7, 2025, and entitled “Natural Salt-Reducing Peptide Composition and Its Use”, the entire contents of which are herein incorporated by reference.

### Sequence Listing Statement

A sequence listing containing the file named “35357620002_Sequence_Listing.xml” which is 8,192 bytes (measured in MS-Windows®) and created on Jan. 7, 2026, contains 4 sequences, is provided herewith via the USPTO's EFS system, and is incorporated herein by reference in its entirety.

### TECHNICAL FIELD

The present disclosure relates to bioengineering technology, in particular to a peptide composition and its use, and more particularly to a natural salt-reducing peptide composition and its use, for the purpose of reducing salt (sodium chloride) content in food and beverage applications without losing the salty sensation.

### BACKGROUND ART

Taste sense serves as a foundation for humans and animals to identify and ingest nutrients. The human taste system reacts with flavor material in ingested food through contact with taste receptors in the mouth and the resulting stimuli are transmitted via the taste nerve to the taste center in the human brain, thereby producing taste perception. Among all these tastes, as one of the essential basic tastes for humans, saltiness serves as a crucial foundation for perceiving the flavor of food. Global mortality risk statistics reveal that high-salt diets are the leading risk factor affecting health. Numerous studies confirm that excessive salt, i.e. sodium chloride ingestion leads to elevated blood pressure. Furthermore, long-term, high-salt diets may increase the incidence of diseases such as gastric cancer, asthma, obesity, and kidney disease. Therefore, it is necessary to reduce salt ingestion in diet. According to research by the European Society of Cardiology, the salt content in food must be lowered, with daily salt ingestion per person kept below 5.00 g. The Dietary Guidelines for Chinese Residents (2022) recommend that the daily salt ingestion for Chinese adults should not exceed 5.00 g.

Numerous salt substitutes, such as non-sodium salts, savory peptides, saltiness-enhancing peptides, and flavor enhancers, have emerged in the public view. These substitutes have demonstrated certain salt-reduction effects in laboratory settings. Non-sodium salt cation substitutes (such as potassium chloride, magnesium chloride, calcium chloride, and potassium lactate) represent the most widely adopted approaches for reducing sodium ingest. These metal salts deliver saltiness and have achieved commercialization. However, salt substitutes can only reduce a part of the sodium content in food, typically not exceeding 15%. Moreover, the replacement cations often impart distinctive flavors, which limits the use of these salt-reduction substitutes in food products, because potassium, magnesium, and many substitute cations stimulate both bitter receptors and salty receptors in the tongue. Flavor enhancers can compensate for the reduced saltiness caused by salt reduction, but this strategy needs to be combined with other salt substitutes to optimize salt crystal shape and particle size, reduce uneven distribution of salt in food matrices, and even restructure food matrices to enhance the perception of saltiness-all of which have been used to address unpleasant tastes. In comparison, salt-reducing peptides serve as a more ideal salt substitute which not only satisfies human taste preferences but also supplements essential amino acids, truly achieving “reducing salt without sacrificing saltiness”.

Salt-reducing peptides refer to bioactive peptides composed of amino acids, extracted from protein-rich raw materials through processes such as enzymatic hydrolysis, which exhibit salt-reducing effects. These peptides may or may not possess saltiness themselves, but when used with salt, they can reduce salt usage without diminishing the perceived saltiness. Based on the food source, salt-reducing peptides can be categorized into animal-derived, plant-derived, and yeast-derived types. Food-derived, salt-reducing peptides originate from fungal and plant proteins can be applied in the field of food salt reduction, offering advantages such as high safety, broad applicability, and better digestibility and utilization by the human body. These peptides represent a highly promising food-derived bioactive peptide.

The molecular mechanisms of salt-reducing peptides remain poorly understood. The perception of saltiness remains the most elusive among all taste sensations. Some specific receptors or ion channels serve as the primary thresholds for recognizing salty substances. Current research focuses on two types of channels: Epithelial Sodium Channel (ENaC), Transient Receptor Potential Vanilloid 1 (TRPV1), and Polycystic Kidney Disease-like Ion Channel (PKD2L1) represent the first category of receptors and ion channels. Transmembrane Channel 4 (TMC4), TRPML3 (MCOLN3), and Kv3.2 have been identified as potential saltiness receptors in the second category of channels. These channels can also be used to screen for salt enhancers. TMC4 is a novel chloride channel sensitive to high NaCl concentrations. This protein contains a transmembrane domain and is expressed in taste buds at the back of the tongue. While TMC4 is not widely recognized as a receptor directly associated with taste perception, a substance activating the TMC4 protein indicates its ability to enhance saltiness, making it useful for identifying and screening salt enhancers.

More specifically about these two types of channels, saltiness stimulants in metal salts primarily consist of compounds formed by cations such as Na⁺, K⁺, Ca²⁺, and Mg²⁺. They dissolve in edible oils or solutions to coat food surfaces or penetrate internal tissues. While chewing, saliva thoroughly mixes with food particles, releasing Na⁺ from the food. Under the action of saliva, Na⁺ is transported to taste buds on the tongue surface, stimulating taste cells and triggering neural transmission to perceive saltiness. Saltiness involves at least two distinct transmission pathways: the amiloride-sensitive pathway and the amiloride-insensitive pathway, each exhibiting different characteristics. The amiloride-sensitive pathway is primarily mediated by the epithelial sodium channel (ENaC), which possesses cation selectivity and is activated mainly by low concentrations of potassium and sodium ions. ENaC is a class of ion channels composed of three distinct subunits (c-subunit, B-subunit, and y-subunit). It plays a crucial role in saltiness perception in rodents. The function of TMC4 type of channels in salt sensing is less clear, exactly how they contribute to the salt perception needs further investigations.

The mechanism of salt-reducing peptide can be partially elucidated through molecular docking. Molecular docking is commonly employed to obtain ligand-receptor complexes, which reflect a static binding pattern. However, due to atomic perturbations, the actual binding process between ligands and receptors is dynamic and constantly evolving. Furthermore, due to limitations in the docking algorithms and scoring functions of existing docking programs, the resulting ligand-receptor complex structures are generally not in a stable state. They can only serve as initial conformations, requiring molecular dynamics simulations to obtain more reliable results. Molecular dynamics simulations are a crucial method for analyzing protein dynamics, enabling the validation and extension of molecular docking results, as well as providing mechanistic explanations for molecular dynamics.

Therefore, to address the aforementioned problems, it is necessary to conduct research on the mechanisms of salt-reducing peptides. Based on this mechanistic research, a natural salt-reducing peptide composition should be selected to better advance related studies on salt reduction effects and health foods.

### SUMMARY

The present disclosure provides a natural salt-reducing peptide composition and its use, which identifies the combination principles and methods for significantly reducing salt consumption. This approach not only substantially decreases salt consumption but also demonstrates clear advantages in taste and food health. This may lead to significant reduction in salt consumption.

To this end, the present disclosure provides a natural salt-reducing peptide composition comprising at least two salt-reducing peptides. Both salt-reducing peptides are capable of binding to an TMC4 receptor and/or an ENaC receptor.

In one or more embodiments, the salt-reducing peptide composition comprises salt-reducing peptides that strongly bind to the TMC4 receptor, as well as salt-reducing peptides that strongly bind to the ENaC receptor.

In one or more embodiments, the salt-reducing peptides are all salt-reducing peptides capable of binding to the TMC4 receptor and the ENaC receptor. Furthermore, when combined with different salt-reducing peptides, bonding forces of salt-reducing peptides EF₁₀ and PP₉ toward TMC4 receptors and ENaC receptors have a high degree of affinity.

In one or more embodiments, the salt-reducing peptide is selected from at least two of EF₁₀, PP₉, and PQ₅. An amino acid sequence of EF₁₀ is shown in SEQ ID NO: 1 as EDEGEQPRPF. An amino acid sequence of PP₉ is shown in SEQ ID NO:2 as PKLLLLPKP. An amino acid sequence of PQ₅ is shown in SEQ ID NO:3 as PHEMQ.

In one example, EF₁₀, PP₉, and PQ₅ are all expressed by protease-deficient Pichia pastoris, as the correct folding and efficient secretion of the salt-reducing peptide within this protease-deficient yeast expression system enhance their expression.

In one or more embodiments, a gene sequence of EF₁₀ is shown in SEQ ID NO:4 as GAGGACGAAG GCGAGCAACC TAGACCTTTT; or a gene sequence of PP₉ is shown in SEQ ID NO:5 as CCTAAATTAT TGTTGTTGCC TAAACCA; or a gene sequence of PQ₅ is shown in SEQ ID NO:6 as CCCCATGAGA TGCAA.

In one or more embodiments, the natural salt-reducing peptide composition comprises EF₁₀ and PP₉. EF₁₀ strongly binds to an ENaC receptor and PP₉ strongly binds to a TMC4 receptor. It is preferred that the natural salt-reducing peptide composition comprises EF₁₀ and PP₉. It is further preferred that the weight ratio of EF₁₀ to PP₉ is (1~3):(1~2), and preferably 1:1.

In one or more embodiments, the natural salt-reducing peptide composition comprises EF₁₀ and PQ₅. EF₁₀ strongly binds to an ENaC receptor and PQ₅ strongly binds to a TMC4 receptor. In this salt-reducing peptide composition, the peptide EF₁₀ exhibits strong binding affinity to the ENaC (epithelial sodium channel) receptor. This strong binding capability enables EF₁₀ to effectively simulate and enhance the perception of saltiness in foods while reducing salt concentration, thereby meeting consumer expectations for flavor. The peptide PQ₅ is then auxiliary-bound to the TMC4 receptor. This binding characteristic of PQ₅ may help regulate or enhance the salt-reducing effect of EF₁₀ by influencing related taste or metabolic pathways, thereby further improving the overall performance of the salt-reducing peptide composition. This auxiliary binding may help regulate or optimize the salt-reducing effect of PP₉ by influencing related taste or metabolic pathways, thereby further enhancing the overall performance of the salt-reducing peptide compositions. It should be noted that the auxiliary binding function of PQ₅ may not be limited to its binding with the TMC4 receptor; it may also interact with other receptors or molecules to jointly promote the enhancement of salt-reducing effects.

Furthermore, although the composition of the peptides PP₉ and PQ₅ is mentioned in one or more embodiments, this does not imply that the salt-reducing peptide composition of the present disclosure is necessarily limited to these two peptides. Depending on specific research and application requirements, other peptides with similar or complementary functions may also be considered for addition to further enrich and enhance the performance and use scope of the salt-reducing peptide composition.

It is preferred that the weight ratio of PP₉ to PQ₅ be (1~3):(1~2), with a preferred ratio of 1:1.

On the other hand, the present disclosure also provides a salt-reducing composition comprising the aforementioned natural salt-reducing peptide composition and edible salt. The weight ratio of the edible salt to the salt-reducing peptide composition is (3~10):1.

In one aspect, the present disclosure provides a use of the aforementioned natural salt-reducing peptide composition or combination in the preparation of low-salt foods or food additives. In one example, the food additive is a salt-reducing agent or a saltiness enhancer.

In another aspect, the present disclosure provides a method for enhancing saltiness or reducing salt content, comprising adding the aforementioned natural salt-reducing peptide composition or combination to salt-containing foods.

Compared to prior art, in one embodiment the natural salt-reducing peptide composition and its use according to the present disclosure employ bioengineering methods to perform multi-copy gene synthesis of the salt-reducing peptides EF₁₀, PP₉, and PQ₅, which are extracted from fermented tofu paste (FSC), yeast extract, Stropharia rugosoannulata, respectively, and conduct codon optimization based on the Pichia pastoris host. After the codon optimization of the natural salt-reducing peptides EF₁₀, PP₉, and PQ₅, gene sequences with higher expression levels for EF₁₀, PP₉, and PQ₅ are obtained. These optimized gene sequences are then constructed into carriers and expressed in the recipient cell of Pichia pastoris (protease-deficient strain MF001-143), thereby enhancing the secretion capacity of the salt-reducing peptides.

Through homology modeling, kinetic simulations are conducted on salt-reducing peptides using the optimal conformation obtained via molecular docking as the initial structure to investigate the interactions between peptides EF₁₀, PP₉, and PQ₅ with ENaC and TMC4. The binding and dissociation processes of peptides with receptors, as well as the stability of the system throughout the simulation are studied. Specifically, at least two of the three salt-reducing peptides are combined and perform molecular docking on the ENaC sodium channel protein and TMC4 chloride channel protein, respectively, so as to investigate the interaction mechanisms between the peptides and the sodium and chloride channel proteins.

Compared to individual peptides EF₁₀, PP₉, or PQ₅, sensory evaluations of these salt-reducing peptide compositions unexpectedly revealed that these compositions demonstrated superior salt-reducing effects while simultaneously enhancing umami flavor. Compared to the individual peptides EF₁₀, PP₉, or PQ₅, the compositions of these salt-reducing peptides-EF₁₀ and PP₉, PP₉ and PQ₅, and EF₁₀, PP₉, and PQ₅-exhibit significant differences in salt reduction and flavor enhancement, demonstrating synergistic effects. Compared to the standard product, the salt-reducing peptide composition of EF₁₀ and PP₉ has a significant difference in terms of salt reduction and enhanced umami. 80% of evaluators report a substantial increase in saltiness with a 33.3% reduction in salt content. When using a combination of salt-reducing peptides in one example, the optimal salt-reducing effect is achieved only when both a strong TMC4 receptor binding to the peptide and an ENaC receptor binding to the peptide are present. The combination of salt-reducing peptides EF₁₀ and PP₉ produces a pronounced synergistic effect. This demonstrates that the salt-reducing peptide composition of the present disclosure holds a great potential as a food additive, significantly reducing salt usage in foods and seasonings.

The salt-reducing peptide composition of the present disclosure not only closely mimics the salty taste of edible salt, providing consumers with an almost indistinguishable sensory experience, but also significantly reduces salt concentration while preserving the original saltiness of food. This characteristic enables the peptide composition of the present disclosure to significantly enhance the health attributes of foods when used as a food additive. It meets the pursuit of modern consumers for low-salt, healthy diets while fully satisfying their expectations for food flavor and taste.

In an aspect, the salt-reducing peptide composition of the present disclosure demonstrates advantages in terms of salt reduction efficacy, flavor retention, and health promotion in food products. Through scientific formulation and selection, the peptide blend reduces sodium ingestion without sacrificing food flavor and taste, offering consumers healthier, more delicious food choices. Furthermore, the method for preparing the natural peptide composition of the present disclosure also offers numerous advantages. For example, the low cost of raw materials reduces production expenses, making this health food additive more accessible for promotion and use. Simultaneously, the high yield of salt-reducing peptides ensures stable production efficiency and product quality. Moreover, the peptide composition according to the present disclosure exhibits high specificity, precisely targeting relevant taste receptors and metabolic pathways to achieve more accurate and efficient salt reduction effects.

In summary, the salt-reducing peptide composition of this disclosure demonstrates advantages in multiple aspects. It not only provides consumers with healthier and more delicious food choices but also offers robust support for the healthy transformation and upgrading of the food industry.

### BRIEF DESCRIPTION OF THE DRAWINGS

To more clearly illustrate the embodiments of the present disclosure or the prior art, the drawings used in the description of the embodiments or the prior art will be briefly introduced below. It is evident that the drawings described below merely represent some embodiments disclosed in the present disclosure. For those skilled in the art, other drawings may be derived from these drawings without requiring creative effort.

FIG. 1 is a diagram showing the PCR identification results of the recombinant transformed strain of gene sequence for the salt-reducing peptide in the embodiment 1 of the present disclosure. Result A shows the PCR identification results of the recombinant transformed strain for the gene sequence of the salt-reducing peptide EF₁₀, M represents the nucleic acid Marker, 1 represents the blank control (water), and 2 represents the negative control. 3 represents the plasmid of the salt-reducing peptide PIC9K-YT-EF₁₀, and 4-7 represent four single colonies randomly selected from the plate. Result B shows the PCR identification results for recombinant transformed strains of the gene sequence for salt-reducing peptide PP₉. M represents the nucleic acid Marker, 1 represents the blank control (water), 2 represents the negative control, 3 represents the plasmid for salt-reducing peptide PIC9K-YT-PP₉, and 4-7 represents four single colonies randomly selected from the plate. Result C shows the PCR identification results for recombinant transformed strains of the gene sequence for the salt-reducing peptide PQ₅. M represents the nucleic acid Marker, 1 represents the blank control (water), 2 represents the negative control, 3 represents the plasmid for the salt-reducing peptide PIC9K-YT-PQ₅, and 4-7 represent four single colonies randomly selected from the plate.

FIG. 2 is an analytical chart showing the purity testing of purified recombinant salt-reducing peptides via HPLC in the embodiment 2 of the present disclosure. Chart A represents the HPLC purity test results for salt-reducing peptide EF₁₀, Chart B represents the HPLC purity test results for salt-reducing peptide PP₉, and Chart C represents the HPLC purity test results for salt-reducing peptide PQ₅.

FIG. 3A is a diagram of the total mass factor of TMC4 in the embodiment 3 of the present disclosure.

FIG. 3B is a tensile diagram of TMC4 in the embodiment 3 of the present disclosure.

FIG. 3C is a three-dimensional diagram of TMC4 in the embodiment 3 of the present disclosure.

FIG. 4A is a 3D diagram of the docking model between the salt-reducing peptide EF₁₀ and the active site of TMC4, along with a 2D diagram showing the interaction details between amino acid residues and the peptide in the embodiment 4 of the present disclosure.

FIG. 4B is a 3D diagram of the docking model between the salt-reducing peptide PP₉ and the active site of TMC4, along with a 2D diagram showing the interaction details between s and the peptide in the embodiment 4 of the present disclosure.

FIG. 4C is a 3D diagram of the docking model between the salt-reducing peptide PQ₅ and the active site of TMC4, along with a 2D diagram showing the interaction details between amino acid residues and the peptide in the embodiment 4 of the present disclosure.

FIG. 5A is a 3D diagram showing the docking model of the salt-reducing peptide EF₁₀ with the active site of ENaC, along with a 2D diagram showing the interaction details between amino acid residues and the peptide in the embodiment 5 of the present disclosure.

FIG. 5B is a 3D diagram of the docking model between the salt-reducing peptide PP₉ and the active site of ENaC, along with a 2D diagram showing the interaction details between amino acid residues and the peptide in the embodiment 5 of the present disclosure.

FIG. 5C is a 3D diagram of the docking model between the salt-reducing peptide PQ₅ and the active site of ENaC, along with a 2D diagram showing the interaction details between amino acid residues and the peptide in the embodiment 5 of the present disclosure.

FIG. 6 is a sensory evaluation radar chart of the salt-reducing peptide in the embodiment 6 of the present disclosure.

FIG. 7 is a chart showing the results of saltiness and umami analysis of the salt-reducing peptide using an electronic tongue in the embodiment 7 of the present disclosure.

FIG. 8 is a statistical significance chart of the results of saltiness and umami analysis of the salt-reducing peptide using an electronic tongue in the embodiment 7 of the present disclosure, * indicates a significant difference and ** indicates a large significant difference.

### DETAILED DESCRIPTION

To enable those skilled in the art to better understand the technical solutions of the present disclosure, the technical solutions in the embodiments of the present disclosure will be described clearly and completely below with reference to the accompanying drawings. Obviously, the described embodiments are only some of the embodiments of the present disclosure, not all of them. Based on the embodiments of the present disclosure, all other embodiments obtained by those skilled in the art without creative labor should fall within the scope of the present disclosure.

Pichia pastoris, the expression host for the Brazzein has been recognized by the U.S. FDA as a GRAS (Generally Recognized as Safe) strain and is also designated as a safe strain for health food production under Chinese law. Additionally, Pichia pastoris possesses the unique ability to grow using methanol as its sole carbon and energy source. Furthermore, Pichia pastoris possesses the ability to undergo high-density fermentation using inexpensive media, tightly regulated and highly potent promoters, excellent post-translational modification and secretion capabilities, and ease of genetic manipulation. These characteristics have established this yeast as a highly successful heterologous protein expression system, widely employed for recombinant protein production-particularly in vaccine manufacturing (with over 5,000 proteins successfully expressed to date).

Unless otherwise specified, “EF₁₀” in the disclosure refers to “Salt-Reducing Peptide EF₁₀”, whose amino acid sequence is EDEGEQPRPF. It is isolated from fermented tofu curd (FSC) by Chen et al. (J Agric Food Chem. 2021 Sep. 8; 69(35):10272-10280. doi: 10.1021/acs.jafc.1c03431. Epub 2021 Aug. 26.) using multiple chromatographic techniques. Sensory evaluation results indicate that the salt perception of 0.4 mg/mL peptide E in a 50 mmol/L NaCl solution is equivalent to that of a 63 mmol/L NaCl solution.

Unless otherwise specified, “PP₉” in the disclosure refers to “Salt-Reducing Peptide PP₉,” whose amino acid sequence is PKLLLLPKP. It is identified by Shan et al. (J Agric Food Chem. 2022 Nov. 30; 70(47):14898-14906. doi: 10.1021/acs.jafc.2c06237. Epub 2022 Nov. 2.) using ultrafiltration to isolate simple taste peptides from yeast extracts. In tests of taste profile description and salt-enhancing effects, PP₉ (acidic taste 0.18 mM) demonstrated a pronounced salt-enhancing effect. Furthermore, S-curve analysis confirmed that PKLLLLPKP exerts an additive effect on salt perception.

Unless otherwise specified, “PQ₅” in the disclosure refers to “salt-reducing peptide PQ₅”, whose amino acid sequence is PHEMQ. It is isolated from Stropharia rugosoannulata by Chen et al. (https://doi.org/10.1016/j.jfca. 2022.104530) from the mushroom Stropharia rugosoannulata. Sensory evaluation and electronic tongue analysis revealed that this peptide segment exhibits good umami activity, with umami thresholds ranging from 0.167 to 0.390 mmol/L.

Unless otherwise specified, “TMC4” or “TMC4 protein” in this disclosure refers to the “TMC4 receptor.”

Unless otherwise specified, “ENaC” or “ENaC protein” in this disclosure refers to the “ENaC receptor”.

Unless otherwise specified, “GalaxyRefine” in this disclosure refers to “a technique for refining protein structures, primarily by optimizing protein conformation through side-chain rearrangement”. This technique significantly improves the accuracy of protein structures, thereby providing more reliable initial models for molecular docking.

The commonly used molecular docking simulation software currently includes SYBYL, MOE, AutoDock, Discovery Studio, and others. Discovery Studio is selected. Compared to common simulation software, Discovery Studio integrates multiple modules such as molecular docking, pharmacophore recognition, virtual screening, quantitative structure-activity relationships (QSAR), combinatorial chemistry, and protein homology modeling. It offers comprehensive and powerful functionality and is widely used in academia and pharmaceutical companies throughout all stages of drug design.

Unless otherwise specified, all reagents and materials mentioned in this disclosure are routinely available through standard commercial channels.

### Embodiment 1. Preparation of Salt-Reducing Peptidase-Deficient Pichia pastoris Strain

The salt-reducing peptides EF₁₀ (SEQ ID NO:1, EDEGEQPRPF), PP₉ (SEQ ID NO:2, PKLLLLPKP), and PQ₅ (SEQ ID NO:3, PHEMQ) are designed with multiple copies. Following codon optimization, the optimized gene sequences for these peptides are synthesized by GenScript Biotech Corporation. The gene sequences of the different peptides after codon optimization are as follows:

The gene sequence of EF₁₀ is shown in SEQ ID NO:4 as GAGGACGAAG GCGAGCAACC TAGACCTTTT. The gene sequence of PP₉ is shown in SEQ ID NO:5 as PKLLLLPKP. The gene sequence of PQ₅ is shown in SEQ ID NO:6 as PHEMQ.

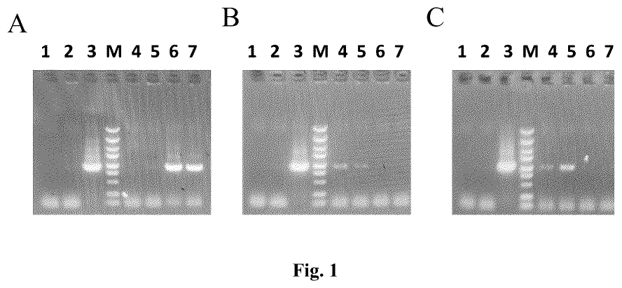

Using the seamless cloning method, the codon optimization gene sequence of EF₁₀, PP₉, and PQ₅ are constructed into the EcoRI restriction site of the pPIC9K plasmid (Thermo Fisher catalog number V17520). The synthesized recombinant vectors pPIC9K-YT-EF₁₀, pPIC9K-YT-PP₉, and pPIC9K-YT-PQ₅ are linearized by SalI restriction enzyme digestion. The linearized product undergoes fragment purification and is subsequently transformed into the protease-deficient Pichia pastoris host strain MF001-143 (detailed information on this strain is documented in Chinese Patent CN202411877517.9, with a deposit number of CCTCC M 20241743) in yeast competent cells. The transformed strains are spread onto YPDS plates containing 4 mg/mL final concentration of geneticin. After incubation at 30° C. for 2-4 days, single colonies are picked for identification. Positive transformants are confirmed by PCR amplification of the target gene in the colonies, with results shown as A, B, and C in FIG. 1.

Ultimately, two transformants are identified for each salt-reducing peptide. Single colonies from these two transformants, validated as positive by PCR, are sequenced by Beijing Qingke Biotechnology Co., Ltd. Shanghai Branch. The initial recombinant bacteria expressing the three salt-reducing peptides are obtained as MF001-143-YT-EF₁₀, MF001-143-YT-PP₉, MF001-143-YT-PQ₅.

### Embodiment 2. Expression Characterization and Purity Testing of Different Recombinant Salt-Reducing Peptides in Protease-Deficient Pichia pastoris Strains

### (1) Preparation of Fermentation Seed Solution

Single colonies of yeast that are streaking-activated and growing well on a YPD (Yeast Extract Peptone Dextrose Medium) plate are picked and inoculated into 50 mL of sterile YPD medium. The YPD is placed in a 30° C. shaker at 250 rpm for overnight cultivation to activate the yeast strain. The yeast seed solution activated by YPD is transferred at a 4% ratio into a 500 mL conical flask containing 100 mL of YPD liquid medium. It is then placed in a 30° C. shaker and cultured overnight at 250 rpm to serve as the seed solution for 5 L fermenter fermentation.

### (2) High-Density Cultivation of Recombinant Yeast Strains in a 5 L Fermenter

The cultured yeast seed solution is inoculated at 8% into a 5 L automatic mechanical stirring aerobic fermenter (pre-sterilized), starting glycerol supplementation for bacteria growth (initial culture medium volume 2 L, sterilized at 121° C. for 30 minutes). Bacteria growth stage: 25% concentrated ammonia solution is used to maintain pH at 5.5, dissolved oxygen is maintained at (30-60)%, temperature is controlled at 30° C., stirring speed is set with an upper limit of 200 rpm and lower limit of 120 rpm, and aeration rate is set to 2 vvm. Once the initial cell density OD600 reaches 400, the glycerol addition should be stopped. When DO (dissolved oxygen) rebounds to over 80%, methanol supplementation is started to induce fermentation. Methanol Feed Stage: Temperature is controlled at 30° C. 25% concentrated ammonia solution is used to maintain pH at 5.5. Rotational speed is controlled at 800 r/min. Gas flow rate is controlled at 4 L/min. Pressure is maintained at 0.05 MPa. Gas flow rate is adjusted to 2 vvm. Methanol is fed using a variable-speed feed method, and dissolved oxygen is maintained between 15-25%. The BLBIO Type B control system software is used for process automation and related data acquisition during fermentation.

### (3) Purification Process for Recombinant Salt-Reducing Peptide in Yeast Strain

The fermentation supernatant obtained after centrifugation and filtration is purified and filtered using the ultrafiltration-nanofiltration membrane filtration system provided by Hangzhou Zhongqi Environmental Protection Technology Co., Ltd. The membrane core employs ceramic membranes with a molecular weight cut-off of 1000 kDa for pre-filtration and sterilization. Subsequently, Synder ceramic membranes with a 30 kDa cut-off are used for ultrafiltration to remove impurities. Finally, an oligofructose gel is utilized for nanofiltration under pressure (0.2-0.3 MPa) to recover soluble salt-reducing peptide. The filtration temperature is maintained at 20° C. After flushing the nanofiltration system with twice its volume of pure water, the recombinant salt-reducing peptide concentrate is obtained. The concentrate is dried and concentrated via freeze-drying. The powder is weighed, sealed, and stored at −20° C. for subsequent saltiness testing of the salt-reducing peptide.

Purity testing of the purified recombinant salt-reducing peptide is performed via HPLC as follows: A Thermo Scientific Vanquish HPLC system equipped with a Luna® 5 μm C4(2) 100 Å, 150×4.6 mm column is used. Stationary phase buffer A (water+0.1% TFA) and mobile phase buffer B (ACN+0.1% TFA) are used. UV detection monitors absorbance at 220 nm. Column temperature is set to 50° C., with an injection volume of 5 μL. The 10-minute gradient program settings are as shown in the following Table 1.

TABLE 1

| Time | Flow (ml/min) | % A | % B |
|---|---|---|---|
| 0.000 | 1 | 80 | 20 |
| 0.100 | 1 | 80 | 20 |
| 7.500 | 1 | 50 | 50 |
| 7.600 | 1 | 80 | 20 |
| 10.000 | 1 | 80 | 20 |

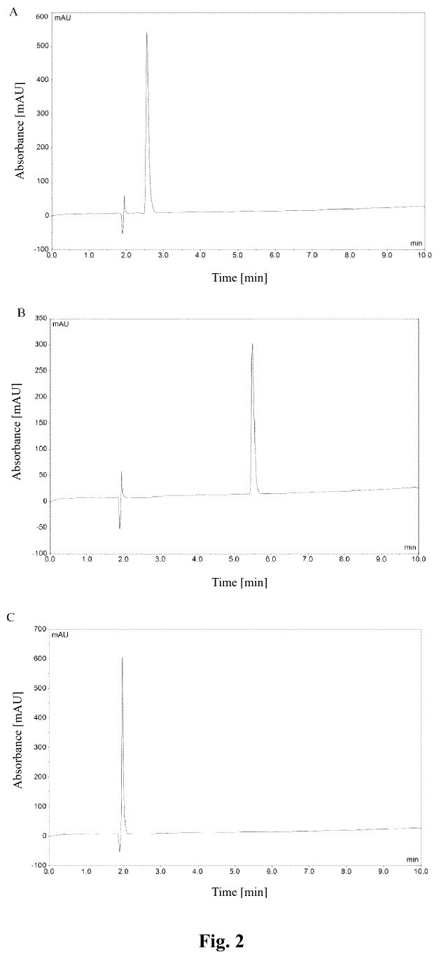

Purity analysis of the purified recombinant salt-reducing peptide is conducted via HPLC, with results shown in A-C of FIG. 2.

As shown in A-C of FIG. 2, among the obtained recombinant salt-reducing peptides, the purity of salt-reducing peptide EDEGEQPRPF (EF₁₀) is 99.0%, the purity of salt-reducing peptide PKLLLLPKP (PP₉) is 99.7%, and the purity of salt-reducing peptide PHEMQ (PQ₅) is 98.2%. The purity of these salt-reducing peptides is sufficient for downstream salt tolerance testing experiments.

### Embodiment 3. Flavor Characterization of Salt-Reducing Peptides

Saltiness receptors exert biological functions by generating cell signaling through interactions with ligands (salty substances). This disclosure employs traditional molecular docking models to analyze the molecular interaction mechanism between salt-reducing peptides and TMC4. By docking potential salt-reducing peptides to the active site of the salt taste receptor protein, the salt-reducing peptides can be further screened. Transmembrane channel-like 4 (TMC4) is selected as the receptor for molecular docking. The amino acid sequence (NP_001138775.2) of the TMC4 protein can be obtained from the NCBI database using the NCBI BLAST tool. The structure of TMC4 is available from the PDB database and is refined by GalaxyRefine (see Nucleic Acids Res. 2013 July; 41(Web Server issue): W384-8. doi: 10.1093/nar/gkt458. Epub 2013 Jun. 3.). The optimized receptor model is subjected to model quality assessment using Ramachandran plots and the overall quality factor (ERRAT). The Ramachandran Plot is primarily used to illustrate the allowed and disallowed conformations of amino acids in proteins or peptides. The most favorable region in the Ramachandran plot indicates that the more amino acids it contains, the more stable the structure is. The disallowed region (blank areas marked with red residues) represents amino acids with unreasonable structures in the ψ-φ (psi-phi). ERRAT distinguishes correct and incorrect protein structures through characteristic atoms. Its overall quality factor is 98.935, and a larger value of this factor indicates the model is closer to high-resolution crystal structures. High degree of similarity to high-resolution crystal structures signifies that the optimized model is more refined.

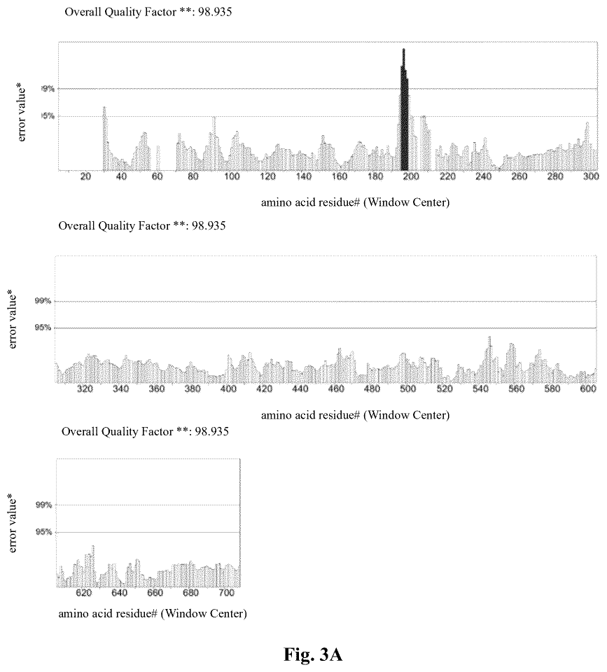

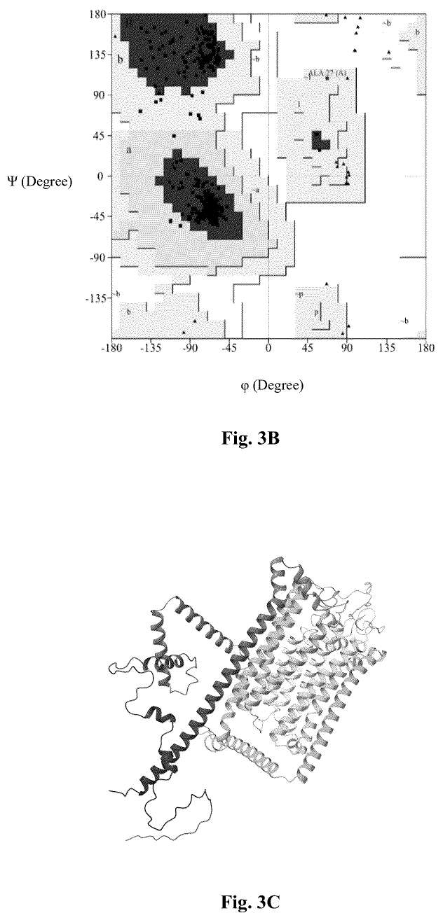

The overall quality factor diagram for TMC4 is shown in FIG. 3A. It analyzes statistical data on non-bonded interactions between different types of atoms and plots the relationship between the error function value and the position of the 9 residues sliding window. This is obtained by comparing and calculating against highly refined statistical data. * denotes two lines plotted on the error axis to indicate the confidence level for regions where values exceeding this error threshold can be rejected. ** represents the percentage of proteins with s below the 95% rejection threshold. A good high-resolution structure typically yields a value of 95% or higher. For lower resolutions (2.5 to 3 Å), the average overall quality factor is approximately 91%. The evaluation results in FIG. 3A indicate that the total quality factor obtained is 98.9%.

The Ramachandran plot is shown in FIG. 3B. Analysis based on FIG. 3B yields the following specific results:

| | Number of | Percentage of |
|---|---|---|
| | amino | amino acid residues |
| | acid | relative to total |
| | residues | residues |
| Amino acid residues in the maximum allowed region [A,B,L] | 602 | 98.2% |
| Amino acid residues in other allowed regions [a,b,l,p] | 10 | 1.6% |
| Amino acid residues in allowed regions [~a,~b,~l,~p] | 1 | 0.2% |
| Amino acid residues in disallowed regions | 0 | 0.0% |
| Number of non-glycine and non-proline residues | 613 | 100% |
| Number of terminal residues (excluding Gly and Pro) | 2 | — |
| Number of glycine residues (displayed as triangles) | 50 | — |
| Number of proline residues | 47 | — |
| Total number of residues | 712 | — |

Based on the analysis of 118 structures, with a resolution of at least 2.0 Angstrom and an R-factor not exceeding 20%, the proportion of high-quality models within the maximum allowed region is projected to exceed 90%.

The evaluation results in FIG. 3B indicate that 98.2% of residues are located in the most favorable region, 1.6% in the allowed region, 0.2% of residues are in the loosely allowed region, and 0.0% of residues are in the disallowed region. In summary, these data confirm that the model is usable.

### Embodiment 4. Investigation of Molecular Interaction Mechanisms Between Salt-Reducing Peptides and TMC4 Based on Molecular Dynamics

The structure of the peptide is constructed using Chem3D 20.0 and preprocessed with Discovery Studio 2019 (DS). This includes the following steps. The Small Molecules module is selected to perform hydrogenation, add the CHARMm force field, and set the charge mode to Momany-Rone. Finally, the file is saved in mol2 format for future use. Simultaneously, the optimized receptor undergoes preprocessing. First, the water molecules and bound ligands are removed. Then, the Clean Protein option under Prepare Protein in the Macromolecules module is selected to refine the amino acid residues of the receptor. The CHARMm force field is also added for energy minimization, Momany-Rone mode is set, and the file is saved in PDB format for future use.

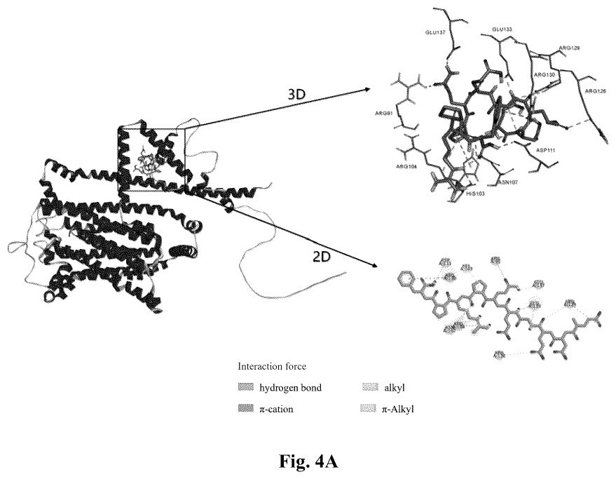

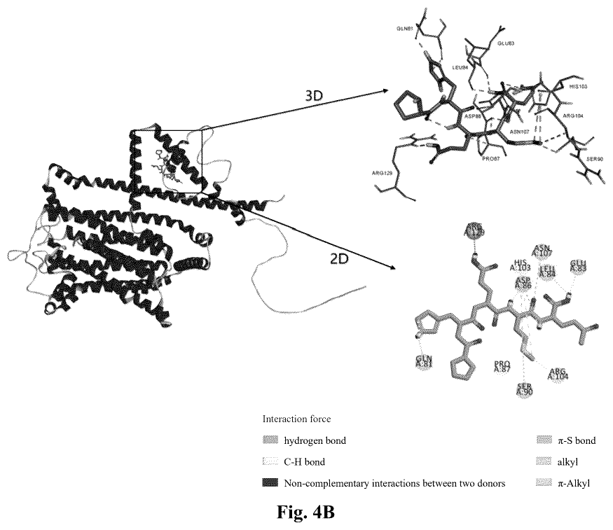

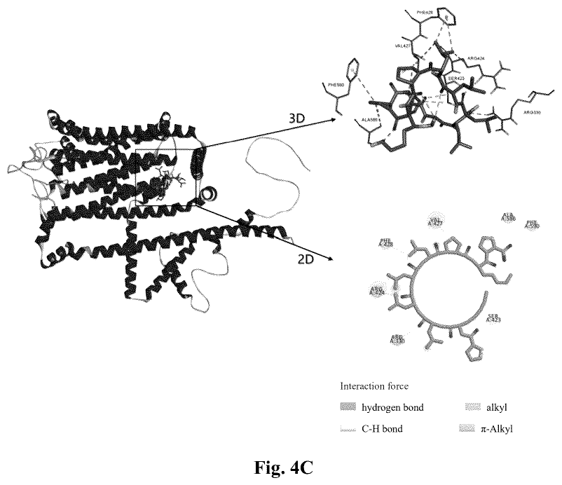

The CDOCKER tool in DS is used to perform semi-flexible molecular docking. The coordinates of the active site sphere are x=17.7065, y=11.7845, z=−14.9743, with a docking radius of 19.7. The CHARMm force field is selected for docking, and the Pose Cluster Radius is set to 0.5 to ensure the docked conformations exhibit maximum diversity. Other parameters are set to default values. Upon completion of the calculation, multiple conformations of a ligand are obtained. Results are analyzed based on binding energy values, with the most stable conformation selected for subsequent molecular dynamics analysis. The trends in the types and quantities of the protein interactions of the three salt-reducing peptides during the simulation are examined. Molecular docking results are shown in FIGS. 4A-C.

Therefore, as shown in FIGS. 4A-C, the theoretical calculations predict the strongest binding to be EF₁₀-7.0 kcal/mol, PQ₅-6.1 kcal/mol, and PP₉-6.7 kcal/mol. These data indicate that EF₁₀ binds most tightly to TMC4 and is the most likely of the three peptides to initially occupy the TMC4 binding site, thereby inducing a conformational change in the receptor.

As shown in FIGS. 4A-C, the data reveals that the protein and the ligand of salt-reducing peptide EF₁₀ which binds most strongly primarily interact via hydrogen bonds, π (Pi) bonds, and salt bridges. When interacting with the TMC4 receptor protein, the ligand forms six hydrogen bonds, two 21 (Pi) bonds, and four salt bridges with the protein. The primary binding sites are: Arg91, Arg104, Asn107, Asp111, Glu133, and Glu137, which are six hydrogen-bonding sites. Two 21 (Pi) bonds are formed at His103 and Arg130; additionally, four salt bridges are formed: at Arg126 with the Glu5 of EF₁₀, at Arg129 with the Glu1 and Gln6 of EF₁₀, and at Lys134 with the Glu3 of EF₁₀.

### Embodiment 5. Investigation of Molecular Interaction Mechanisms Between Salt-Reducing Peptides and ENaC Based on Molecular Dynamics

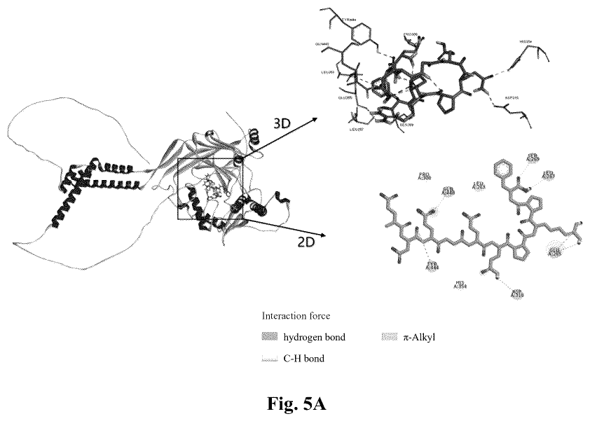

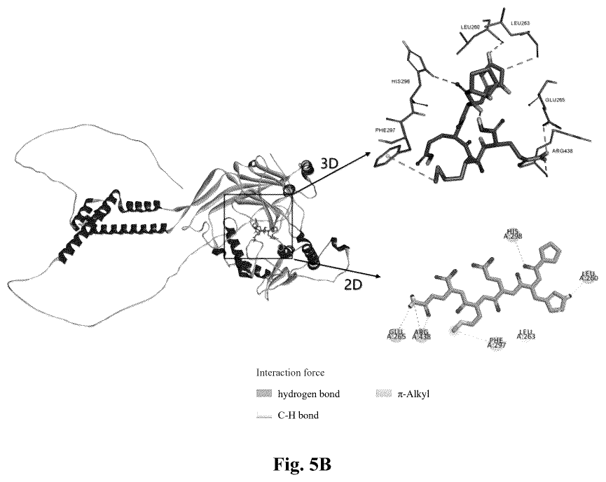

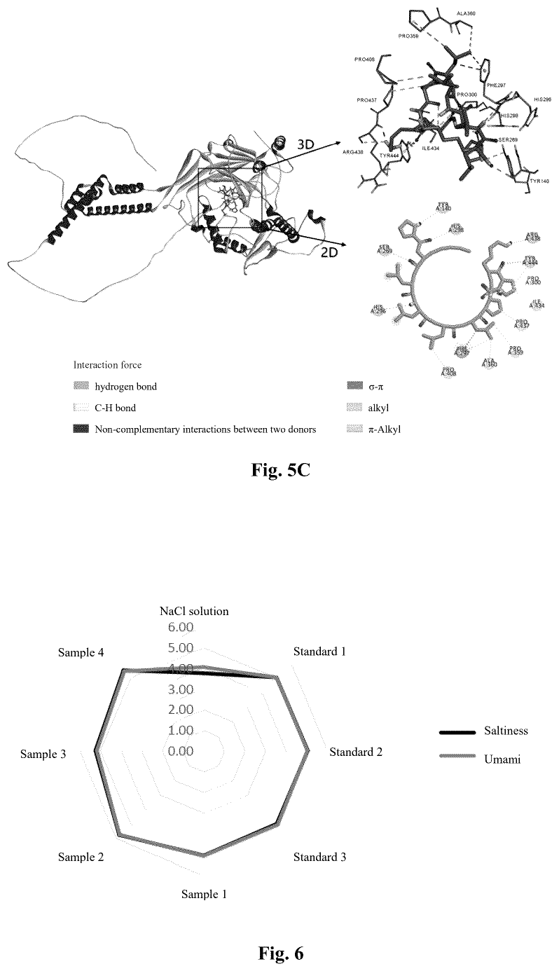

The ENaC plays a crucial role in salt perception in rodents. This disclosure employs molecular docking models to revalidate the molecular interaction mechanism between salt-reducing peptides and the ENaC. By docking potential salt-reducing peptides to the active site of the salt taste receptor protein, further screening of these peptides can be conducted. The ENaC (amiloride-sensitive sodium channel subunit alpha isoform 3 [Homo sapiens]) is selected as the receptor for molecular docking. The amino acid sequence (NP_001153047.1) of the ENaC protein can be obtained from the NCBI database using the NCBI BLAST tool. The structure of ENaC can be obtained from the PDB database. Molecular docking is performed using the same steps and methods as in embodiment 4, with the results shown in FIGS. 5A-C.

It can be seen from FIGS. 5A-C that the binding strengths of the three salt-reducing peptides to ENaC also differ, being EF₁₀-6.1 kcal/mol, PQ₅-6.9 kcal/mol, and PP₉-8.7 kcal/mol respectively. By analyzing the trends in the types and quantities of the protein interactions of the three salt-reducing peptides during the simulation process, the data reveals that the protein and the ligand of salt-reducing peptide PP₉ which binds most strongly primarily interact via hydrogen bonds and salt bridges. When interacting with the ENaC receptor protein, the ligand forms six hydrogen bonds and one σ-π(sigma-π) bond with the protein. The primary binding sites are: Tyr140, Ser269, His296, His298, Arg438, and Tyr444. And a 6-71 bond is also formed at Pro300.

Therefore, combining the results from the embodiment 4 with the energy analysis and binding strength analysis, it is evident that PP₉ exhibits the strongest binding to ENaC, while EF₁₀ exhibits the strongest binding to TMC4. To verify whether the presumed three combinations of salt-reducing peptide could provide superior salt-reducing effects and to explore the existence of synergistic interactions, we conducted sensory evaluation trials on different combinations. This approach aims to validate our hypotheses through practical evaluation and determine the optimal combination of ingredients for achieving the greatest salt-reducing effect.

### Embodiment 6. Sensory Evaluation of Salt-Reducing Peptides

1. Sample Preparation: The salt-reducing peptide and NaCl are prepared as 100 mL mixed solutions at different concentrations. At first, a solution of 3 mg/mL NaCl without any additives is designated as the ‘NaCl solution’ and serves as the negative control. Then, the standard solution and test samples are prepared according to the following ratios.

(i) A 100 mL mixture solution is prepared using 3 mg/mL NaCl+0.1 mg/mL salt-reducing peptide EDEGEQPRPF (EF₁₀) and used as Standard 1 (J Agric Food Chem. 2021 Sep. 8; 69(35):10272-10280. doi: 10.1021/acs.jafc.1c03431. Epub 2021 Aug. 26.).

(ii) A mixture solution of 100 mL is prepared using 3 mg/mL NaCl+0.1 mg/mL salt-reducing peptide PKLLLLPKP (PP₉) and used as Standard 2.

(iii) A mixture solution of 100 mL is prepared using 3 mg/mL NaCl+0.1 mg/mL salt-reducing peptide PHEMQ (PQ₅), and used as Standard 3.

(iv) mixture solution is prepared using 2 mg/mL NaCl+0.1 mg/mL salt-reducing peptide EDEGEQPRPF (EF₁₀)+0.1 mg/mL salt-reducing peptide PHEMQ (PQ₅), and used as Sample 1.

(v) A mixture solution is prepared using 2 mg/mL NaCl+0.1 mg/mL salt-reducing peptide EDEGEQPRPF (EF₁₀)+0.1 mg/mL salt-reducing peptide PKLLLLPKP (PP₉), and used as Sample 2.

(vi) A mixture solution is prepared using 2 mg/mL NaCl+0.1 mg/mL salt-reducing peptide PHEMQ (PQ₅)+0.1 mg/mL salt-reducing peptide PKLLLLPKP (PP₉), and used as Sample 3.

(vii) A mixture solution is prepared using 2 mg/mL NaCl+0.1 mg/mL salt-reducing peptide EDEGEQPRPF (EF₁₀)+0.1 mg/mL salt-reducing peptide PHEMQ (PQ₅)+0.1 mg/mL salt-reducing peptide PKLLLLPKP (PP₉), and used as Sample 4.

2. Sample Testing: Ten persons (five males and five females, aged 22-35) who have experience in appearance, flavor, and texture assessment of salt-reducing peptides are selected for comparative evaluation. Manual sensory evaluation is conducted in a sensory analysis room maintained at 22.5±2.5° C. with standard lighting conditions. Each participant must refrain from alcohol abuse and smoking. Participants work in separate, independent spaces to ensure tasting results remain unbiased. To guarantee impartiality, each sample group is assigned three random numeric codes, and all samples are presented to participants in a randomized sequence. During the evaluation process, participants rinse their mouths with purified water, then hold the sample in their mouths for 2 minutes before spitting it out. Based on predetermined reference standards, participants will score samples using a 10-point system: 0-2 points indicate extremely weak saltiness/umami 4-6 points indicate moderate saltiness/umami 6-8 points indicate relatively strong saltiness/umami 8-10 points indicate very intense saltiness/umami.

The test results are shown in Table 2-3 and FIG. 6 below.

TABLE 2
Sensory Evaluation for Saltiness of Salt-reducing Peptide Compositions

| | NaCl | Standard | Standard | Standard | Sample | Sample | Sample | Sample |
|---|---|---|---|---|---|---|---|---|
| Staff | solution | 1 | 2 | 3 | 1 | 2 | 3 | 4 |
| Male | 3 | 5 | 5.4 | 5 | 6 | 7 | 5 | 5.5 |
| Female | 4 | 5 | 6 | 4.6 | 5 | 6 | 4 | 5.6 |
| Male | 3 | 5 | 5.5 | 5.1 | 6 | 6.5 | 5.2 | 5.2 |
| Male | 3.6 | 5 | 5 | 5 | 5.5 | 5.5 | 4.8 | 6.2 |
| Female | 3.5 | 5 | 5.1 | 5.1 | 6.2 | 5.7 | 6.3 | 6 |
| Female | 3.1 | 5 | 5.2 | 4.7 | 4.3 | 5 | 4.5 | 4.5 |
| Female | 4.6 | 5 | 4 | 4.8 | 3 | 4.8 | 4.8 | 4.6 |
| Male | 4.4 | 5 | 4.3 | 4.6 | 3.5 | 4.5 | 6 | 4 |
| Male | 4.5 | 5 | 5 | 5.2 | 5.3 | 6.2 | 6.2 | 6.5 |
| Female | 3.7 | 5 | 5 | 6 | 6 | 6.6 | 5.6 | 6.8 |
| Average | 3.74 | 5 | 5.05 | 5.01 | 5.08 | 5.78 | 5.24 | 5.49 |
| value | | | | | | | | |

TABLE 3
Sensory Evaluation for Umami of Salt-reducing Peptide Compositions

| | NaCl | Standard | Standard | Standard | Sample | Sample | Sample | Sample |
|---|---|---|---|---|---|---|---|---|
| Staff | solution | 1 | 2 | 3 | 1 | 2 | 3 | 4 |
| Male | 4 | 5 | 6.3 | 5.5 | 5.8 | 7 | 5.2 | 5.1 |
| Female | 4.1 | 5 | 6.3 | 5.1 | 5 | 6.2 | 4.6 | 5.3 |
| Male | 3.9 | 5 | 5.7 | 5.3 | 6 | 6.2 | 5.4 | 5.4 |
| Male | 3.8 | 5 | 5.5 | 5.2 | 5 | 5.3 | 4.6 | 6 |
| Female | 4.2 | 5 | 4.6 | 6 | 6 | 5.8 | 6 | 6.3 |
| Female | 4 | 5 | 4 | 4.7 | 4.6 | 5.5 | 4.8 | 4.8 |
| Female | 3.5 | 5 | 4.5 | 5 | 4 | 4.8 | 4.6 | 4.8 |
| Male | 4.4 | 5 | 4.6 | 4.8 | 4 | 4.3 | 5.2 | 4.4 |
| Male | 3.9 | 5 | 4 | 4.5 | 5.1 | 6 | 5.8 | 6 |
| Female | 4.6 | 5 | 5.3 | 4.2 | 5.5 | 6.4 | 5.5 | 6.2 |
| Average | 4.04 | 5 | 5.08 | 5.03 | 5.1 | 5.75 | 5.17 | 5.43 |
| value | | | | | | | | |

As shown in Table 2-3 and in FIG. 6, the four salt-reducing peptide compositions of the present disclosure exhibit a distinct umami-enhancing effect compared to standards 1, 2, and 3, along with increased saltiness. The ranking of saltiness-umami flavor is as follows: Sample 2>Sample 4>Sample 3>Sample 1>Standard 2>Standard 3>Standard 1>NaCl solution.

When comparing Sample 2 with the Standard, 80% of participants report a significant increase in saltiness despite a 33.3% reduction in salt content. This demonstrates that the salt-reducing peptide composition of the present disclosure holds a great potential as a food additive capable of substantially reducing salt usage in foods and seasonings.

### Embodiment 7. Electronic Tongue Analysis of Salt-Reducing Peptides

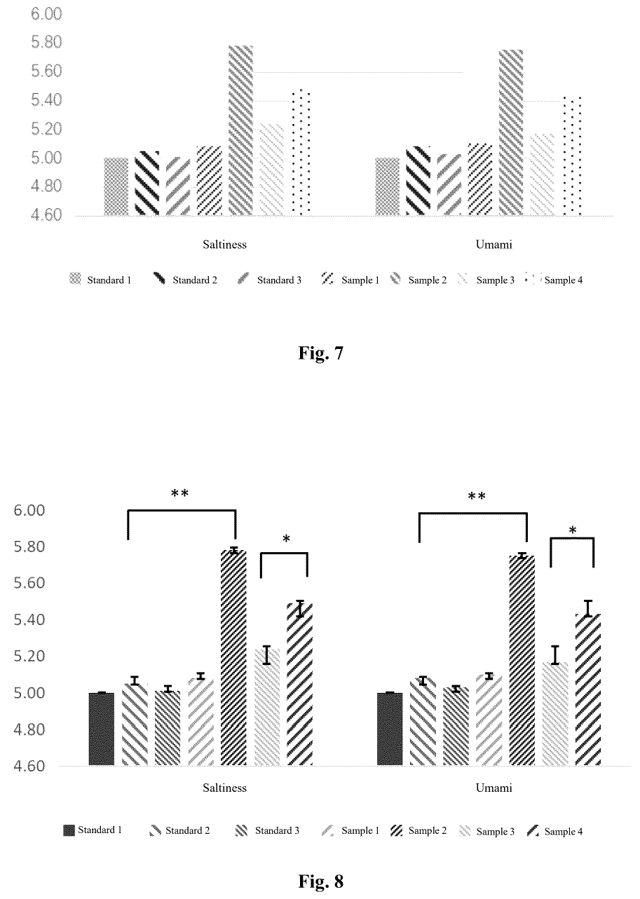

The electronic tongue is a rapid, accurate, and unbiased method for evaluating flavor. The salt-reducing effect of salt-reducing peptide compositions is assessed using the ThinkSenso&Senso electronic tongue. The saltiness score is measured using the saltiness of a mixed solution containing 3 mg/mL NaCl+0.1 mg/mL salt-reducing peptide EDEGEQPRPF (EF₁₀) as the saltiness standard. Saltiness scores are measured for Standards 2 and 3, and Samples 1, 2, 3, and 4. Each sample is measured in parallel 10 times, and the average value is taken. The results are shown in FIGS. 7-8.

As shown in FIGS. 7-8, the saltiness-umami ranking for the four sample groups is as follows: Sample 2>Sample 4>Sample 3>Sample 1>Standard 2>Standard 3>Standard 1. The results from the electronic tongue align with those from sensory evaluation.

It is found that the four salt-reducing peptide compositions demonstrated superior salt-enhancing effects compared to Standards 1, 2, and 3, along with enhanced umami characteristics. Sample 1 shows superior effects in both salt-reducing and umami-enhancing compared to the salt-reducing peptides EF₁₀ and PQ₅. Sample 2 shows superior effects in both salt-reducing and umami-enhancing compared to the salt-reducing peptides EF₁₀ and PP₉, with significant differences indicating that the combination of EF₁₀ and PP₉ not only reduces salt content and enhances umami but also exhibits a significant synergistic effect. Sample 3 shows superior effects in both salt-reducing and umami-enhancing compared to the salt-reducing peptides PQ₅ and PP₉, with significant differences indicating that the combination of PQ₅ and PP₉ not only reduces salt content and enhances umami but also exhibits a synergistic effect. Sample 4 shows superior effects compared to standards 1, 2, and 3, with significant effects, and its performance also surpasses that of samples 1 and 3.

Furthermore, most importantly, it is also found that when using a combination of salt-reducing peptides, the optimal salt-reducing effect is achieved when both a TMC4 receptor binding to the peptide and an ENaC receptor binding to the peptide are present. For example, the effects of Sample 2 and Sample 4 are superior to those of Sample 1 and Sample 3. However, it is found that when the strongest TMC4 receptor binding to the peptide (EF₁₀) and the strongest ENaC receptor binding to the peptide (PP₉) are present simultaneously, Sample 2 shows a better effect than other combinations. This suggests that when these two salt-reducing peptides are present, they exhibit strong binding affinity, thereby producing a highly synergistic effect.

After comparing samples with different combinations of salt-reducing peptides, it is found that when all three peptides are present simultaneously (i.e., Sample 4), the effect is slightly less than that of Sample 2, which contains only two of the peptides. Nevertheless, it still showed a superior effect compared to samples containing other combinations (Samples 1 and 3) or a single peptide (such as EF₁₀). It is hypothesized that this may be due to PQ₅ binding weakly to both associated receptors, thereby interfering with the expected effects of the other two peptides (potentially those used in combination with PQ₅). Thus, the overall performance of Sample 4 failed to surpass that of Sample 2. On the other hand, for the single-peptide sample containing only EF₁₀, its binding to the ENaC receptor initiates a single signaling pathway. This may prevent it from effectively influencing the two associated metabolic pathways, resulting in a salt-reducing effect inferior to the carefully balanced combination formulation in Sample 2.

In summary, the analysis indicates that interactions between peptides and their binding capacity to specific receptors are crucial for achieving optimal salt-reducing effects. The salt-reducing peptide composition in Sample 2 may have achieved optimal synergistic effects, thereby demonstrating the best salt-reducing performance. Although Sample 4 is affected by the binding of PQ₅ to receptors, it still achieved good salt-reducing performance due to the synergistic effects of EF₁₀ and PP₉. It is necessary to gain a deeper understanding of the interaction mechanisms among individual peptides to identify optimal compositions. This provides insights for future development of highly efficient and precise salt-reducing peptide compositions.

The following numbered paragraphs provide embodiments of the present disclosure:
1. A natural salt-reducing peptide composition comprising salt-reducing peptides EF10 and PP9, wherein bonding forces of salt-reducing peptides EF10 and PP9 toward TMC4 receptors and ENaC receptors have a high degree of affinity; an amino acid sequence of EF10 is shown in SEQ ID NO:1 as EDEGEQPRPF; an amino acid sequence of PP9 is shown in SEQ ID NO:2 as PKLLLLPKP.
2. The natural salt-reducing peptide composition as recited in paragraph 1, wherein both EF10 and PP9 are expressed by protease-deficient Pichia pastoris.
3. The natural salt-reducing peptide composition as recited in paragraph 1, wherein a gene sequence of EF₁₀ is shown in SEQ ID NO:4 as GAGGACGAAG GCGAGCAACC TAGACCTTTT; a gene sequence of PP9 is shown in SEQ ID NO:5 as CCTAAATTAT TGTTGTTGCC TAAACCA.
4. The natural salt-reducing peptide composition as recited in any one of paragraphs 1 to 3, wherein EF₁₀ strongly binds to an ENaC receptor and PP9 strongly binds to a TMC4 receptor.
5. The natural salt-reducing peptide composition as recited in paragraph 4, wherein the weight ratio of EF₁₀ to PP9 is (1-3):(1-2).
6. The natural salt-reducing peptide composition as recited in paragraph 5, wherein the weight ratio of EF₁₀ to PP9 is 1:1.
7. A salt-reducing composition comprising a natural salt-reducing peptide composition as claimed in any one of claims 1 to 6 and an edible salt, the weight ratio of the edible salt to the salt-reducing peptide composition is (3~10):1.
8. A use of the natural salt-reducing peptide composition as recited in any one of paragraphs 1 to 6, or the salt-reducing composition as recited in paragraph 7, in a preparation of low-salt foods or food additives.
9. The use as recited in paragraph 8, wherein the food additive is a salt-reducing agent or a saltiness enhancer.
10. A method for enhancing saltiness or reducing salt content, comprising adding the natural salt-reducing peptide composition as claimed in any one of claims 1 to 6 or the salt-reducing composition as recited in paragraph 7 to salt-containing food.

It is apparent to those skilled in the art that the present disclosure is not limited to the details of the exemplary embodiments described above, and that it is capable of implementing the present disclosure in other specific forms without departing from the spirit or essential features of the present disclosure. Accordingly, the embodiments are to be regarded as exemplary and non-limiting, and the scope of the present disclosure is limited by the appended claims and not by the foregoing description, and is therefore intended to encompass all variations falling within the meaning and scope of the equivalent elements of the claims. Any accompanying markings in the claims should not be regarded as limiting the claims involved.

In addition, it should be understood that although the specification is described in accordance with the embodiments, not each embodiment contains only one independent technical solution, and the specification is recited in such a manner only for the sake of clarity, and the person skilled in the art should take the specification as a whole, and the technical solutions in the embodiments may be combined appropriately to form other embodiments that can be understood by the person skilled in the art.

What is claimed is:

1. A natural salt-reducing peptide composition comprising salt-reducing peptides EF₁₀ and PP₉, wherein bonding forces of salt-reducing peptides EF₁₀ and PP₉ toward TMC4 receptors and ENaC receptors have a high degree of affinity; an amino acid sequence of EF₁₀ is shown in SEQ ID NO:1 as EDEGEQPRPF; an amino acid sequence of PP₉ is shown in SEQ ID NO:2 as PKLLLLPKP.

2. The natural salt-reducing peptide composition as claimed in claim 1, wherein both EF₁₀ and PP₉ are expressed by protease-deficient Pichia pastoris.

3. The natural salt-reducing peptide composition as claimed in claim 1, wherein a gene sequence of EF₁₀ is shown in SEQ ID NO:4 as GAGGACGAAG GCGAGCAACC TAGACCTTTT; a gene sequence of PP₉ is shown in SEQ ID NO:5 as CCTAAATTAT TGTTGTTGCC TAAACCA.

4. The natural salt-reducing peptide composition as claimed in claim 1, wherein EF₁₀ strongly binds to an ENaC receptor and PP₉ strongly binds to a TMC4 receptor.

5. The natural salt-reducing peptide composition as claimed in claim 4, wherein the weight ratio of EF₁₀ to PP₉ is (1-3):(1-2).

6. The natural salt-reducing peptide composition as claimed in claim 5, wherein the weight ratio of EF₁₀ to PP₉ is 1:1.

7. The natural salt-reducing peptide composition as claimed in claim 1 in combination with an edible salt to form a combination composition, the weight ratio of the edible salt to the salt-reducing peptide composition is from about 3:1 to about 10:1.

8. A method of using the natural salt-reducing peptide composition as claimed in claim 1 comprising using the natural salt-reducing peptide composition in a preparation of a low-salt food or a food additive.

9. The method as claimed in claim 8, wherein the food additive is a salt-reducing agent or a saltiness enhancer.

10. A method for enhancing saltiness or reducing salt content, comprising adding the natural salt-reducing peptide composition as claimed in claim 1 to a salt-containing food.


SEQUENCE LISTING (4 sequences)

SEQ ID NO: 1 - protein, 10 aa, Synthetic construct
EDEGEQPRPF

SEQ ID NO: 2 - DNA, 30 bp, synthetic construct
  source 1..30: mol_type = other DNA
gaggacgaaggcgagcaacctagacctttt

SEQ ID NO: 3 - DNA, 27 bp, synthetic construct
  source 1..27: mol_type = other DNA
cctaaattattgttgttgcctaaacca

SEQ ID NO: 4 - DNA, 15 bp, synthetic construct
  source 1..15: mol_type = other DNA
ccccatgagatgcaa